(19) United States
(12) Patent Application Publication
(10) Pub. No.: US 2022/0410134 A1
(43) Pub. Date: Dec. 29, 2022

(54) ALKYLATION CATALYST COMPOSITION AND RELATED METHODS

(71) Applicant: University of Kansas, LAWRENCE, KS (US)

(72) Inventors: Mark Brandon Shiflett, Lawrence, KS (US); Rajkumar Kore, Lawrence, KS (US); Aaron M. Scurto, Oskaloosa, KS (US)

(21) Appl. No.: 17/775,540

(22) PCT Filed: Nov. 13, 2020
(86) PCT No.: PCT/US20/60403

§ 371 Date: May 9, 2022

Related U.S. Application Data
(60) Provisional application No. 62/935,900, filed on Nov. 15, 2019.

(51) Int. Cl. B01J 31/02; C07C 2/62

(52) U.S. Cl. CPC B01J 31/0284; B01J 31/0298; C07C 2/62; B01J 2231/44; C07C 2531/025

(57) ABSTRACT

An alkylation catalyst composition is provided which comprises an acid, an aromatic, and a third component selected from the group consisting of a base capable of forming an ionic liquid with the acid; and an ionic liquid. An alkylation process is also provided which comprises combining the alkylation catalyst composition with a feedstock under conditions to produce an alkylate product for a motor fuel additive. The alkylate product produced by the alkylation process is also provided.

### CROSS-REFERENCE TO RELATED APPLICATIONS

The present application claims priority to U.S. provisional patent application No. 62/935,900 that was filed Nov. 15, 2019, the entire contents of which are incorporated herein by reference.

### BACKGROUND

Alkylation of isobutane with olefins is a key process in the production of gasoline. Conventionally, strong liquid acids such as anhydrous HF or H₂SO₄ are used to alkylate isobutane with butene. However, such conventional processes present significant safety and environmental issues due to the handling of large quantities of spent H₂SO₄ and hazardous HF. As an alternative, solid acid catalysts have also been developed. However, these catalysts suffer from other drawbacks, including rapid deactivation, which results in low product yield and loss of reaction selectivity. Ionic liquids have also been used in isobutane alkylation processes, but these have exhibited limited activity.

### SUMMARY

The present disclosure provides catalyst compositions and methods of making and using the catalyst compositions, including in catalyzing isobutane/butene alkylation for motor fuels.

In embodiments, an alkylation catalyst composition is provided which comprises an acid, an aromatic, and a third component selected from the group consisting of a base capable of forming an ionic liquid with the acid; and an ionic liquid. An alkylation process is also provided which comprises combining the alkylation catalyst composition with a feedstock under conditions to produce an alkylate product for a motor fuel additive. The alkylate product produced by the alkylation process is also provided.

In embodiments, an alkylate product is provided which comprises branched alkanes comprising trimethylpentane and dimethylhexane and has a percentage of C8 alkanes of at least 95% and a T/D ratio of at least 10.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1C show illustrative cations which may be used to form an ionic liquid for use in the present catalyst compositions.

FIG. 1D shows illustrative cations which may be used to form an ionic liquid for use in the present catalyst compositions.

FIG. 1E shows illustrative bases for use in the present catalyst compositions.

FIG. 2 shows illustrative anions which may be used to form an ionic liquid for use in the present catalyst compositions.

FIG. 3 shows illustrative acids for use in the present catalyst compositions.

FIG. 4 shows illustrative aromatics for use in the present catalyst compositions.

FIG. 5 is a schematic of a reaction system for carrying out an alkylation reaction using the present catalyst compositions.

### DETAILED DESCRIPTION

The present disclosure provides catalyst compositions and methods of making and using the catalyst compositions. When used in isobutane/butene alkylation, at least some embodiments of the catalyst compositions achieve extremely high conversion values (e.g., >99%), C8 selectivity (e.g., >90%), and ratio of trimethylpentane/dimethylhexane (T/D ratio) (e.g., >25). By contrast, under the same conditions, catalysts such as [HN₂₂₂][Al₂Cl₇] (with or without CuCl) achieve a much lower C8 selectivity (30-40%) and T/D ratio (e.g., 0.4-3).

The present catalyst compositions are multicomponent ionic systems which are typically liquids near room temperature (e.g., 20 to 25° C.). The catalyst compositions comprise an acid, an aromatic, and a third component. The third component may either be an ionic liquid or a base which forms, in situ, an ionic liquid when combined with the acid in the catalyst composition. The acid is generally present in an amount in excess of the aromatic and the third component. Without wishing to be bound to any particular theory, it is believed that the three components (or ions generated from the three components) may associate to form a molecular complex having unique, synergistic properties, as distinguished from a simple mixture of the individual components. Thus, in the present disclosure, terms such as “ternary complex,” “clathrate,” and the like may be used to describe this molecular complex. However, such terms are not intended to limit the scope of structural form of the molecular complex or catalyst composition. In the present disclosure, the term “ternary mixture” may also be used in reference to the catalyst composition. Finally, as demonstrated in the Examples, below, it has been found that the component types and amounts of each component can be selected to tune the alkylation behavior, including to achieve the superior C8 selectivity and T/D ratios in isobutane/butene alkylation noted above.

A variety of ionic liquids/bases, acids, and aromatics may be used in the catalyst compositions. The particular combination of components may be selected to achieve alkylation behavior, e.g., desired conversion, C8 selectivity, and T/D ratio. Specific combinations are illustrated in the Examples below, but these are not intended to be limiting. Other combinations may be formed using the guidance provided below.

Ionic Liquids

In embodiments, the third component of the present catalyst compositions is an ionic liquid. Various ionic liquids may be used. As used in the present disclosure, “ionic liquid” includes organic salts that are fluid at or below a temperature of about 100° C. However, the phrase can also refer to certain ionic compounds or ionic solids that dissolve when combined with the acid to form the catalyst composition or the term “ionic solid” or “ionic compound” may be used to refer to such solids/compounds.

Representative examples of ionic liquids suitable for use herein are included among those that are described in sources such as J. Chem. Tech. Biotechnol., 68:351-356 (1997); Chem. Ind., 68:249-263 (1996); J Phys. Condensed Matter, 5: (supp 34B): 899-8106 (1993); Chemical and Engineering News, Mar. 30, 1998, 32-37; J Mater. Chem., 8:2627-2636 (1998); Chem. Rev., 99:2071-2084 (1999); and WO 05/113,702 (and references cited therein), each of which is by this reference incorporated herein for the purpose of the ionic liquids disclosed therein.

Many ionic liquids are formed by reacting a nitrogen-containing heterocyclic ring, preferably a heteroaromatic ring, with an alkylating agent (e.g., an alkyl halide) to form a quaternary ammonium salt, and performing ion exchange or other suitable reactions with various Lewis acids or their conjugate bases to form the ionic liquid. Some ionic liquids are formed by reacting N-, P-, and S-compounds with a Bronsted acid to quatemize the heteroatom. Examples of suitable heteroaromatic rings include substituted pyridines, imidazole, substituted imidazole, pyrrole and substituted pyrroles. These rings can be alkylated with virtually any straight, branched or cyclic C1-20 alkyl group, but the alkyl groups are preferably C1-16 groups. Various trialkylphosphines, thioethers and cyclic and non-cyclic quaternary ammonium salts may also be used for this purpose. Ionic liquids suitable for use herein may also be synthesized by salt metathesis, by an acid-base neutralization reaction, or by quatemizing a selected nitrogen-containing compound. The synthesis of other ionic liquids suitable for use herein is described in U.S. Pat. No. 8,715,521, which is by this reference incorporated in its entirety as a part hereof for all purposes. Ionic liquids may also be obtained commercially from several companies such as Merck (Darmstadt, Germany), BASF (Mount Olive N.J.), Fluka Chemical Corp. (Milwaukee Wis.), and Sigma-Aldrich (St. Louis Mo.).

Ionic liquids suitable for use herein comprise a cation and an anion. A variety of cations and anions may be used. Either or both of the ions may be fluorinated. The ionic liquid may include more than one type of cation, more than one type of anion, or both. However, the ionic liquid may include a single type of cation and a single type of anion. When the ionic liquid includes a single type of cation and a single type of anion, however, this does not preclude some amount of ion exchange with other ions in the catalyst composition (derived from other components of the catalyst composition).

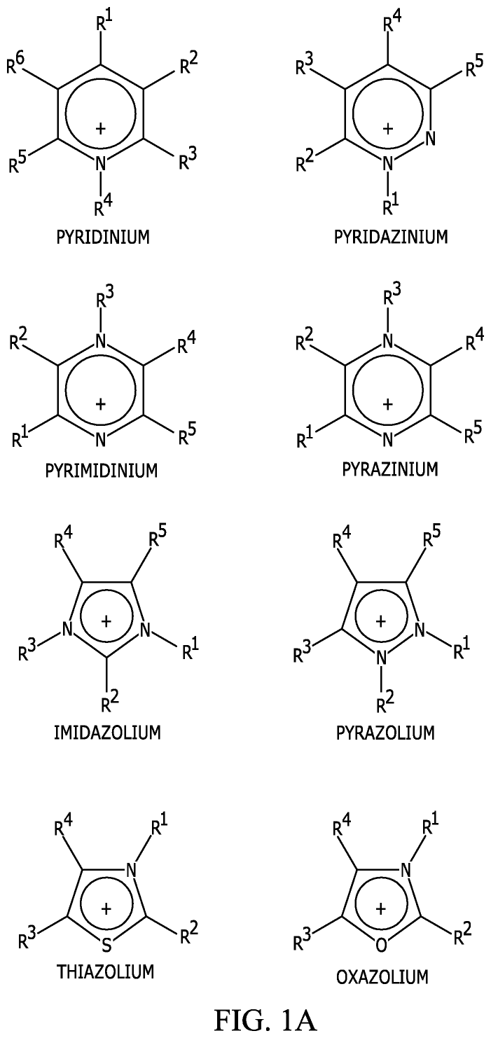

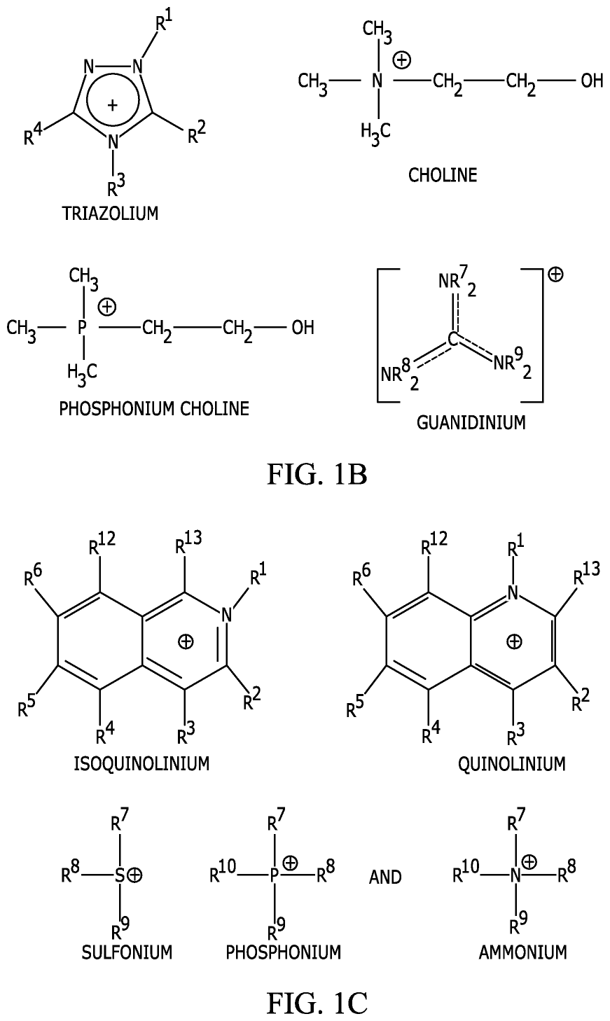

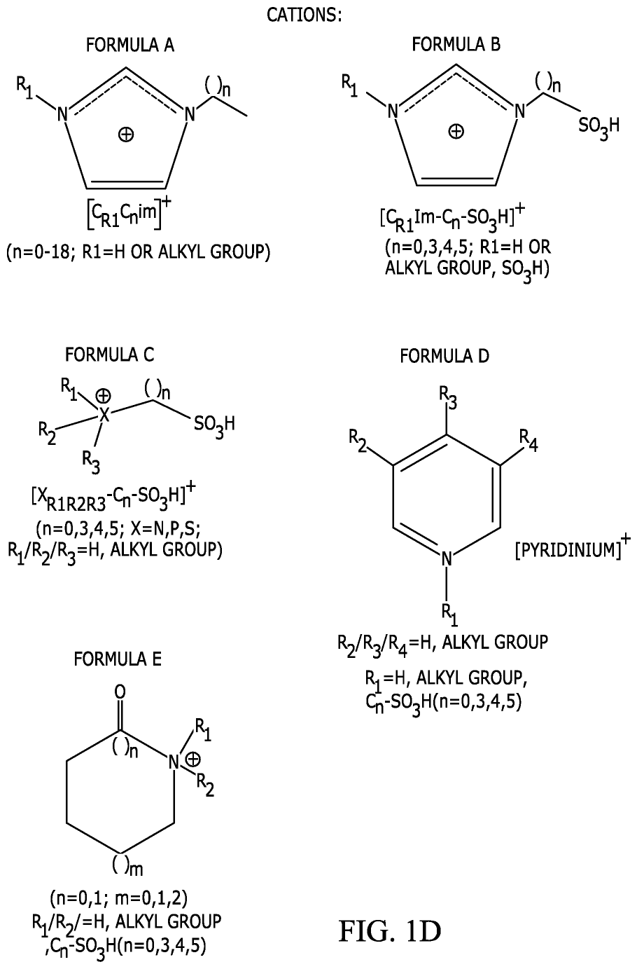

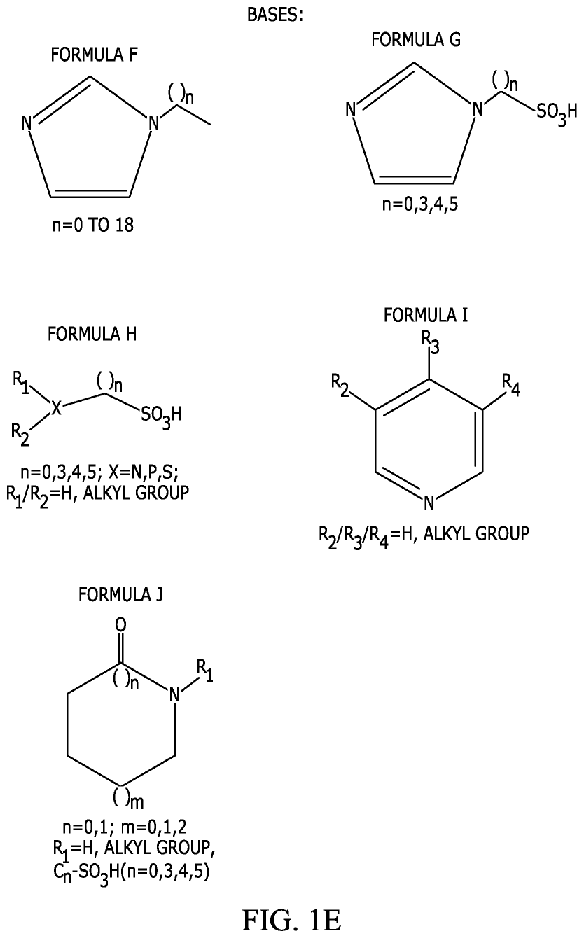

In embodiments, the cation is selected from the group consisting of cations represented by the structures of the formulae shown in FIGS. 1A-1C. In these formulae, the following provisos apply:

(a) R¹, R², R³, R⁴, R⁵, R⁶, R¹² and R¹³ are independently selected from the group consisting of:

### (i) H;

(ii) halogen such as F;
(iii) —CH₃, —C₂H₅, or C₃ to C₂₅ straight-chain, branched or cyclic alkane or alkene groups, optionally substituted with at least one member selected from the group consisting of Cl, Br, F, I, OH, NH₂SH, and SO₃H;
(iv) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(v) C₆ to C₂₅ unsubstituted aryl, or C₆ to C₂₅ unsubstituted heteroaryl, groups having one to three heteroatoms independently selected from the group consisting of O, N, Si and S, wherein the unsubstituted aryl or unsubstituted heteroaryl may be bonded to the structure via an alkyl (e.g., —CH₂—) spacer group;
(vi) C₆ to C₂₅ substituted aryl, or C₆ to C₂₅ substituted heteroaryl, groups having one to three heteroatoms independently selected from the group consisting of O, N, Si and S; wherein the substituted aryl or substituted heteroaryl may be bonded to the structure via an alkyl (e.g., —CH₂—) spacer group; and wherein said substituted aryl or substituted heteroaryl has one to three substituents independently selected from the group consisting of:
(A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
(B) OH,
(C) NH₂, and
(D) SH; and
   (vii) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4;

(b) R⁷, R⁸, R⁹, and R¹⁰ are independently selected from the group consisting of:

(i) —CH₃, —C₂H₅, or C₃ to C₂₅ straight-chain, branched or cyclic alkane or alkene groups, optionally substituted with at least one member selected from the group consisting of Cl, Br, F, I, OH, NH₂, SH and SO₃H;
(ii) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(iii) C₆ to C₂₅ unsubstituted aryl, or C₆ to C₂₅ unsubstituted heteroaryl, groups having one to three heteroatoms independently selected from the group consisting of O, N, Si and S; and
(iv) C₆ to C₂₅ substituted aryl, or C₆ to C₂₅ substituted heteroaryl, groups having one to three heteroatoms independently selected from the group consisting of O, N, Si and S, and wherein the substituted aryl or substituted heteroaryl group has one to three substituents independently selected from the group consisting of:
(A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
(B) OH,
(C) NH₂, and
(D) SH; and
   (v) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4; and

(c) optionally, at least two of R¹, R², R³, R⁴, R⁵, R⁶, R⁷, R⁸, R⁹, and R¹⁰ can together form a cyclic or bicyclic alkyl or alkenyl group.

In embodiments, the ionic liquid comprises a cation selected from one or more members of the group consisting of pyridinium, pyridazinium, pyrimidinium, pyrazinium, imidazolium, pyrazolium, thiazolium, oxazolium, triazolium, phosphonium, ammonium, benzyltrimethylammonium, choline, cholinium, dimethylimidazolium, guanidinium, phosphonium choline, lactam, sulfonium, tetramethylammonium, and tetramethylphosphonium.

In embodiments, the ionic liquid comprises an anion selected from one or more members of the group consisting of: [CH₃CO₂]⁻, [HSO4]⁻, [CH₃OSO₃]⁻, [C₂H₅OSO₃]⁻, [CH₃C₆H₄SO₃]⁻([TSO]⁻), [AlCl4]⁻, [Al₂Cl₇]⁻, [ZnCl₄]²⁻, [Zn₂Cl₆]²−, [Zn₃Cl₈]²⁻, [FeCl₄]⁻, [GaCl₄]⁻, [Ga₂Cl₇]⁻, [InCl₄]⁻, [In₂Cl₇]⁻, [CO₃]²⁻, [HCO₃]⁻, [NO₂]⁻, [NO₃]⁻, [SO₄]²⁻, [PO₃]³⁻, [HPO₃]²⁻, [H₂PO₃]⁻, [PO₄]³⁻, [HPO₄]²⁻, [H₂PO₄]⁻, [HSO₃]⁻, [CuCl₂]⁻, Cl⁻, Br⁻, I⁻, SCN⁻, carborates optionally substituted with alkyl or substituted alkyl; carboranes optionally substituted with alkylamine, substituted alkylamine, alkyl or substituted alkyl; and a fluorinated anion.

In embodiments, the ionic liquid comprises an anion selected from one or more members of the group consisting of aminoacetate, ascorbate, benzoate, catecholate, citrate, dimethylphosphate, formate, fumarate, gallate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃SO₃]⁻, [CH₃OSO₃]⁻, [C₂H₅OSO₃]⁻, [CH₃C₆H₄SO₃]⁻, [AlCl4]⁻, [Al₂Cl₇]⁻, [ZnCl₄]²⁻, [Zn₂Cl₆]²⁻, [Zn₃Cl₈]²⁻, [FeCl₄]⁻, [GaCl₄]⁻, [Ga₂Cl₇]⁻, [InCl₄]⁻, [In₂Cl₇]⁻, [CO₃]²⁻, [HCO₃]⁻, [NO₂]⁻, [NO₃]⁻, [SO₄]²⁻, [PO₃]³⁻, [HPO₄]²⁻, [H₂PO₄]⁻, [HSO₃]⁻, [CuCl₂]⁻, Cl⁻, Br⁻, I⁻, SCN⁻, [BF₄]⁻, [PF₆]⁻, [SbF₆]⁻, [CF₃SO₃]⁻, [HCF₂CF₂SO₃]⁻, [CF₃HFCCF₂SO₃]⁻, [CHF₂CF₂CF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, [(CF₂HCF₂SO₂)₂N]⁻, [(CF₃CFHCF₂SO₂)₂N]⁻, [N(CN)₂]⁻, F⁻, and anions represented by the structure of the following formula, [R₁₁COO]⁻, wherein R¹¹ is selected from the group consisting of:

(i) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH;
(ii) —CH₃, —C₂H₅, or C₃ to C₁₀ straight-chain, branched or cyclic alkane or alkene groups that contain one to three heteroatoms selected from the group consisting of O, N, Si and S, and are optionally substituted with at least one member selected from the group consisting of Cl, Br, F, I, OH, NH₂ and SH;
(iii) C₆ to C₁₀ unsubstituted aryl, or C₆ to C₁₀ unsubstituted heteroaryl, groups having one to three heteroatoms independently selected from the group consisting of O, N, Si and S; and
(iv) C₆ to C₁₀ substituted aryl, or C₆ to C₁₀ substituted heteroaryl, groups having one to three heteroatoms independently selected from the group consisting of O, N, Si and S; and wherein the substituted aryl or substituted heteroaryl group has one to three substituents independently selected from the group consisting of:
(A) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH,
(B) OH,
(C) NH₂, and
(D) SH.

In embodiments, the cation of the ionic liquid is selected from an imidazolium, an ammonium, a phosphonium, a sulfonium, a pyridinium, and a lactam. The cation may be protic or aprotic. The proton in the protic cation may be from a —SO₃H group. Illustrative imidazolium, ammonium, phosphonium, sulfonium, pyridinium, and lactam cations are shown in FIG. 1D. In embodiments, the cation of the ionic liquid is selected from the group consisting of cations represented by the structures of the formulae shown in FIG. 1D, i.e., Formulae A-E. In these formulae, the provisos noted in FIG. 1D apply.

The anion of the ionic liquid may be a sulfonate. The sulfonate may have the formula [R—SO₃]⁻, wherein R is an alkyl group or an aryl group. The alkyl group may be a linear alkyl group in which the number of carbons may range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F. Aryl groups may be unsubstituted or substituted as described above with respect to alkyl groups. However, substituted aryl groups also refer to an unsubstituted monocyclic aryl group in which one or more carbon atoms are bonded to an alkane. The alkane may be linear, have various numbers of carbon, and may be unsubstituted or substituted as described above with respect to alkyl groups.

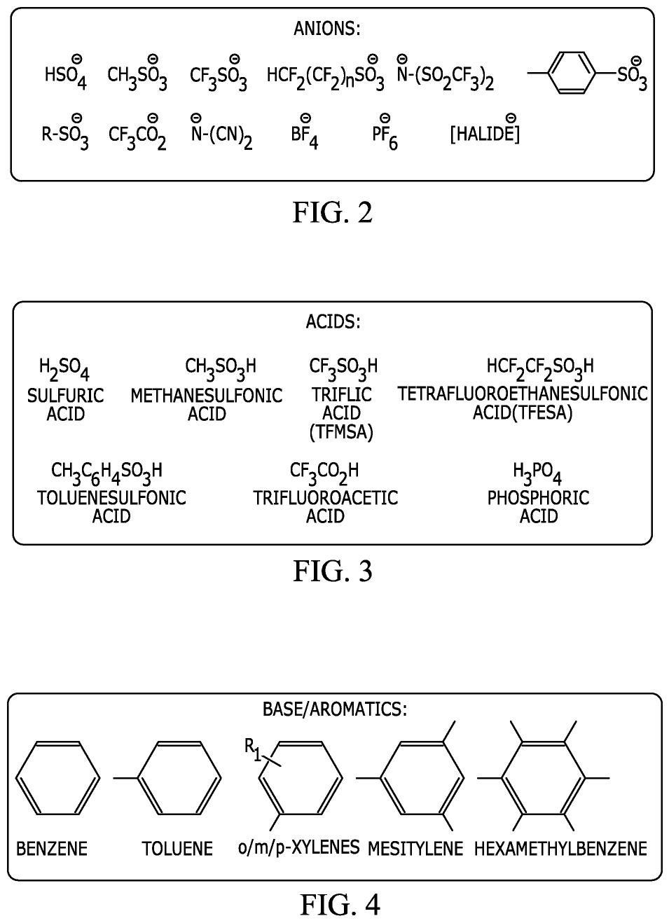

The anion may be a carboxylate. The carboxylate may have the formula [R—CO₂]⁻, wherein R is an alkyl group as described above with respect to sulfonate. This means that fluoroalkane carboxylates are encompassed, e.g., R may be CF₃, HCF₂CF₂, CF₃HFCCF₂, etc. The carboxylate (or fluoroalkane carboxylate) may be a dicarboxylate, a tricarboxylate, a tetracarboxylate, etc. Other anions which may be used include [HSO₄]⁻, dicyanamide; and inorganic anions such as [BF₄]⁻, [PF₆]⁻, and a halide. Illustrative anions are shown in FIG. 2. In [HCF₂(CF₂)ₙSO₃]⁻, n may be 0, 1, 2, or 3.

Ionic liquids disclosed in the following references may also be used: U.S. Pat. Nos. 8,771,626; 8,779,220; 8,808,659; U.S. Pat. Pub. No. 20100331599; U.S. Pat. Nos. 7,432,408; 9,914,674; U.S. Pat. Pub. No. 20160289138; U.S. Pat. Pub. No. 20140113804; U.S. Pat. Pub. No. 20160167034; U.S. Pat. Pub. No. 20150315095; and U.S. Pat. Nos. 9,567,273; 9,346,042; 9,260,668; 9,096,487; 8,692,048; 8,653,318; 8,633,346; 8,569,561; 8,552,243; and 7,285,698. Each of these is by this reference incorporated herein for the purpose of the ionic liquids disclosed therein.

In the ionic liquids, various relative amounts of the cation(s) and anion(s) may be used. In embodiments, the molar ratio of the cation:anion is in the range of from 1:1 to 4:1.

Other illustrative specific ionic liquids are provided in the Examples, below.

Known methods may be used to prepare ionic liquids. Other ionic liquids may be commercially available. Illustrative methods for synthesizing ionic liquids are described in the Examples, below.

Bases

In embodiments, the third component of the present catalyst compositions is a base which forms, in situ, an ionic liquid (or an ionic compound or an ionic solid) when combined with the acid in the catalyst composition. Thus, any base which generates any of the cations described in “Ionic Liquids,” above, upon combination with the acid of the catalyst composition may be used. By way of illustration, the base may be an imidazole, an ammonia, a phosphine, a sulfide, a pyridine, or a lactam. The base be selected from the group of compounds having any of the formulae shown in FIG. 1E, i.e., Formulae F-J. In these formulae, the alkyl group may be as defined above with respect to sulfonate in “Ionic Liquids.”

The catalyst composition may comprise different types of bases. However, a single type of base may also be used.

Acids

Various acids may be used in the present catalyst compositions, including combinations of different types of acids. However, a single type of acid may also be used. Mineral acids may be used, e.g., sulfuric acid, phosphoric acid, hydrofluoric acid, hydrochloric acid.

Sulfonic acids may be used. The sulfonic acid may have the formula R—SO₃H, wherein R is an alkyl group or an aryl group. The alkyl group may be linear, branched, or cyclic and may have a number of carbons in a range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br, and I. Aryl groups may be unsubstituted or substituted as described above with respect to alkyl groups. However, substituted aryl groups also refer to an unsubstituted monocyclic aryl group in which one or more carbon atoms are bonded to an alkane. The alkane may be linear, branched, or cyclic, have various numbers of carbon atoms, and may be unsubstituted or substituted as described above with respect to alkyl groups.

Carboxylic acids may be used. The carboxylic acid may have the formula R—CO₂H, wherein R is an alkyl or an aryl group as described above with respect to sulfonic acid. Strong metal acids such as AlCl₃, GaCl₃, InCl₃, FeCl₃, ZnCl₃, CuSO₄, etc., may also be used. Illustrative specific acids are shown in FIG. 3.

Although in some embodiments sulfuric acid (H₂SO₄) may be used, in other embodiments, the acid is not sulfuric acid and the catalyst composition is free of sulfuric acid. Throughout the present disclosure, the term “free” means that the amount of the relevant component is zero or sufficiently close to zero to have no material effect on the properties of the catalyst composition.

Aromatics

Various aromatics may be used in the present catalyst compositions, including combinations of different types of aromatics. However, a single type of aromatic may also be used.

The aromatic may be monocyclic having one or more unfused aromatic rings. Each aromatic ring may have various members, e.g., a 5-membered ring, a six-membered ring, etc. Monocyclic aromatics may be unsubstituted, by which it is meant the aromatic contains only carbon and hydrogen and no heteroatoms. Unsubstituted monocyclic aromatics have a single aromatic ring. Monocyclic aromatics may be substituted, by which it is meant an unsubstituted aromatic in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br; 0; N; etc. However, substituted monocyclic aromatics also refer to an unsubstituted monocyclic aromatic in which one or more carbon atoms are bonded to an unsubstituted or substituted alkane or another unsubstituted or substituted monocyclic aromatic. The alkane may be linear or branched, have various numbers of carbon atoms, and may be unsubstituted or substituted as described above with respect to alkyl groups in “Acids.” Thus, monocyclic aromatics include benzene, biphenyl, triphenyl, furan, pyridine, pyrrole, etc. (each which may be unsubstituted or substituted).

The monocyclic aromatic may have the formula C₆R₆, wherein each R is independently selected from hydrogen, a halogen, and an alkyl group. The alkyl group may be linear or branched have various numbers of carbon atoms and may be unsubstituted or substituted as described above with respect to alkyl groups in “Acids.” Illustrative such monocyclic aromatics are shown in FIG. 4.

Polycyclic aromatics may be used. Polycyclic aromatics have fused aromatic rings (e.g., two, three, etc. rings). Each ring may have various members and may be unsubstituted or substituted as described for monocyclic aromatics. Naphthalene, anthracene, phenanthrene, benzofuran are illustrative polycyclic aromatics.

Similar to the bases described above, it is noted that the aromatic used in the catalyst composition may be one which forms, in situ, an ionic liquid when combined with the acid in the catalyst composition.

Each of the three components may be present at various amounts in the present catalyst composition. However, as shown in the Examples, below, it has been found that the alkylation behavior of the catalyst composition is particularly sensitive to the amount of the aromatic component. For example, in isobutane/butene alkylation, the C8 selectivity and the T/D ratio can be tuned over a relatively large range by relatively small adjustments of the amount of the aromatic component. In embodiments, the aromatic component is present at an amount in a range of from of 0.1 wt % to 25 wt %. This includes from 1 wt % to 15 wt %, 1 wt % to 10 wt %, from 3 wt % to 9 wt %, or from 5 wt % to 8 wt %. Specific, illustrative amounts are provided in the Examples, below. Throughout the present disclosure, “wt %” refers to weight percent. The weight percent of the aromatic component is ((weight of the aromatic component)/(combined weight of the ionic liquid (or base) and the acid))*100.

The balance of the catalyst composition may be made up of the ionic liquid (or the base) and the acid and various relative amounts of the ionic liquid (or the base) and the acid may be used. Again, these amounts may be selected to provide desired alkylation behavior. In embodiments, the ionic liquid/base is present at an amount in a range of from 2 wt % to 80 wt % and the acid is present at an amount in a range of from 98 wt % to 20 w %. This includes embodiments in which the ionic liquid/base is present at an amount in a range of from 5 wt % to 60 wt %, from 5 wt % to 30 wt % or 5 wt % to 20 wt % and the acid is present at an amount in a range of from 95 wt % to 40 wt %, 95 wt % to 70 wt % or 95 wt % to 80 wt %, respectively. Here, the weight percent of the ionic liquid/base (or acid) is ((weight of the ionic liquid/base (or acid))/(combined weight of the ionic liquid/base and the acid))*100. Specific, illustrative amounts are provided in the Examples, below.

An amount of water may be present in the catalyst composition. However, in embodiments, the catalyst composition consists or consists essentially of the ionic liquid (or the base), the acid, and the aromatic. In such embodiments, however, more than one type of each component may be used, i.e., more than one type of ionic liquid/base, more than one type of acid, and/or more than one type of aromatic. In other such embodiments, a single type of each component may be used.

The present catalyst compositions may be made by combining the ionic liquid/base and the acid at the desired relative amounts followed by adding the aromatic at the desired amount. The synthesis may be carried out while stirring and under room temperature. Other details are provided in the Examples, below.

As noted above, ion exchange may occur between the various components of the present catalyst compositions. In addition, there may be some overlap between compounds suitable for the various components, e.g., some compounds may be suitable as a base and an aromatic. However, at least in embodiments, catalyst compositions described as comprising an “ionic liquid,” an “acid”, and an “aromatic” refer to compositions in which separate and distinct chemical compounds are combined in forming the composition regardless of how the various ions may rearrange/associate in the composition. E.g., in such embodiments, a chemically distinct ionic liquid, a chemically distinct acid, and a chemically distinct aromatic are combined to form the catalyst composition. Similarly, at least in embodiments, catalyst compositions described as comprising a “base,” an “acid”, and an “aromatic” refer to compositions in which distinct chemical compounds are combined in forming the composition, regardless of subsequent ion associations. E.g., in such embodiments, a chemically distinct base, a chemically distinct acid, and a chemically distinct aromatic are combined to form the catalyst composition.

The present catalyst compositions may be used in an alkylation process to provide an alkylate product for a motor fuel additive. In embodiments, such a method comprises combining a feedstock and any of the disclosed catalyst compositions under conditions to produce the alkylate product. The feedstock may comprise an alkane and an olefin. The alkane may have four or more carbons, i.e., a C4 alkane. The alkane may be an isoalkane. The olefin may have four carbons, i.e., a C4 olefin, but olefins having other numbers of carbons may be used, e.g., C3, C5, C6. The olefin may be an iso-olefin. The feedstock may comprise isobutane and butene, e.g., 2-butene. Other alkanes and olefins may be used, e.g., propane, pentane, propene, isobutene, 1-butene, trans-2-butene, cis-2-butene, pentenes, amylenes, etc. The feedstock may comprise different types of alkanes and different types of olefins. However, a single type of alkane and a single type of olefin may also be used. Under the appropriate conditions, the disclosed catalyst compositions can catalyze the conversion of the alkane(s) and olefin(s) of the feedstock into an alkylate product for a motor fuel additive comprising a mixture of branched alkanes. The method may further comprise recovering the alkylate product from the reaction mixture (the combined feedstock and catalyst composition).

The conditions under which alkylation occurs refer to parameters such as the amount of the catalyst composition used, the amount of feedstock used, the reaction temperature, the reaction time, and the reaction pressure. These parameters may be adjusted to provide desired alkylation behavior, e.g., a desired conversion, C8 selectivity, and T/D ratio. In embodiments, the amount of the catalyst composition used in the alkylation reaction is in a range of from 0.5 g to 5 g, from 1 g to 4 g, or from 1 g to 3 g. In embodiments, the amount of feedstock used in the alkylation reaction is in a range of from 1 mL to 10 mL, from 1 mL to 5 mL, or from 2 mL to 5 mL. Of course, the amount of the catalyst composition and the amount of feedstock may be scaled up as necessary for commercial processes, e.g., from 3 kg/gal to 8 kg/gal catalyst composition to feedstock may be used In embodiments, the reaction temperature is in a range of from 0° C. to 20° C., from 1° C. to 15° C., from 3° C. to 8° C., or from 5° C. to 8° C. In embodiments, the reaction time is in a range of from 0.5 min to 10 min, from 1 min to 8 min, or from 1 min to 5 min. Similarly, the reaction time may be adjusted in scaled-up commercial processes. In embodiments, the reaction pressure is in the range of from 1 bar to 60 bar. For alkane/olefin alkylation reactions, the conditions may also include the alkane/olefin ratio, which may be from 1:1 to 15:1. Illustrative conditions and combinations of conditions are provided in the Examples, below.

A variety of reactor systems may be used to carry out the alkylation process, including batch, semi-continuous, continuous, and spray reactor systems. An illustrative system is shown in FIG. 3.

The present catalyst compositions and alkylation reactions may be characterized as being capable of achieving certain properties or results, including a percent conversion, a percent C8 selectivity, and a T/D ratio. Known methods may be used to calculate these values, e.g., see U.S. Pat. Pub. No. 20100331599, which by this reference is incorporated herein in its entirety. In embodiments, the conversion is at least 95%, at least 99%, at least 99.5%, or at least 100%. In embodiments, the C8 selectivity is at least 75%, at least 80%, at least 85%, at least 90%, or at least 98%. In embodiments, the T/D ratio is at least 10, at least 15, at least 20, at least 25, at least 30, or at least 60. These properties may be referenced with respect to a particular set of reaction conditions, e.g., a set of reaction conditions as set forth in the Examples, below. These reaction conditions may include a pure isobutane and 2-butene feedstock.

The alkylation processes may also be characterized by the purity of the recovered alkylate product. The alkylate product may be recovered relatively quickly from the reaction mixture and the recovered alkylate product may be free of the catalyst composition and any of its components.

The alkylate product formed using the disclosed processes are also encompassed by the present disclosure. Gasoline comprising the alkylate product is also encompassed.

### EXAMPLE

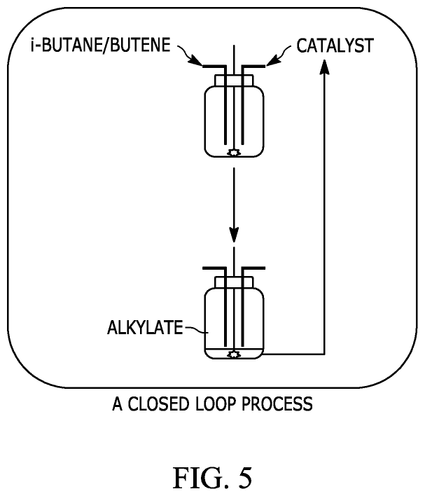

These Examples describe the preparation of illustrative ionic liquids using the cations and anions shown in FIGS. 1D and 2 as well as the preparation of illustrative catalyst compositions comprising the ionic liquids and the acids and aromatics of FIGS. 3 and 4, respectively. Also described is the preparation of illustrative catalyst compositions comprising bases of FIG. 1E and the acids and aromatics of FIGS. 3 and 4, respectively. These catalyst compositions as well as comparative catalyst compositions were tested in isobutane/butene alkylation using the reactor system shown in FIG. 5. The results are shown in Table 1, below.

### Example 1. Preparation of Ionic Liquids

### Example 1-I Preparation of N-Sulfonic Acid Diethyl Ammonium Hydrogen Sulfate [HN₂₂—SO₃H][HSO₄] IL

In a 500 mL round bottom flask, equipped with a stir bar, diethylamine (20 g, 0.27 mol) was reacted with chlorosulfonic acid (35.02 g, 0.30 mol) in dry dichloromethane solvent (40 mL). After addition, the reaction mixture was stirred for 12 h. The dichloromethane solvent was removed under reduced pressure, yielding a brown slurry. The slurry was washed twice with dry diethyl ether and dried under vacuum, yielding light brown solid of diethylsulfamic acid (N₂₂—SO₃H). For the synthesis of [HN₂₂—SO₃H][HSO₄] IL, a solid diethylsulfamic acid (7.66 g, 0.05 mol) was placed in a 20 mL screw top borosilicate glass vial and liquid sulfuric acid (H₂SO₄; 4.95 g, 0.05 mol) was added dropwise. After addition of the reactants, the reaction mixture was stirred at room temperature for 4 h, giving a brown liquid IL [HN₂₂—SO₃H][HSO₄].

### Example 1-II: Preparation of N-Sulfonic Acid Diethyl Ammonium Tetrafluoroethane Sulfonate [HN₂₂—SO₃H][TFES] IL

In a 20 mL screw top borosilicate glass vial, equipped with a stir bar, an amount of diethylsulfamic acid (7.66 g, 0.05 mol) was placed and then liquid tetrafluoroethanesulfonic acid (TFESA) (9.15 g, 0.05 mol) was added dropwise. After addition, the reaction mixture was stirred at room temperature for 4 h, giving a brown liquid IL [HN₂₂—SO₃H][TFES].

### Example 1-III: Preparation of N-Methyl Imidazolium Hydrogen Sulfate [C₁im][HSO₄] IL

In a 50 mL round bottom flask, equipped with a stir bar, an amount of N-methyl imidazole (4.11 g, 0.05 mol) was placed and then liquid H₂SO₄ acid (4.95 g, 0.05 mol) was added dropwise at 5° C. After addition, the reaction mixture was stirred at 80° C. for 4 h, giving a liquid IL [C₁im][HSO₄].

### Example 1-IV: Preparation of N,N-Disulfonic Acid Imidazolium Hydrogen Sulfate [Im(-SO₃H)₂][HSO₄] IL

In a 500 mL round bottom flask, equipped with a stir bar, imidazole (6.80 g, 0.10 mol) was reacted with chlorosulfonic acid (23.77 g, 0.205 mol) in dry dichloromethane solvent (400 mL). The reaction mixture was stirred for 12 h to obtain a biphasic system containing a lower IL layer and an upper dichloromethane layer. The dichloromethane solvent was decanted, yielding a brown liquid. The IL layer was washed twice with dichloromethane and dried under vacuum, yielding a brown liquid of N,N-disulfonic acid imidazolium chloride [Im(-SO₃H)₂][Cl]. For the synthesis of N,N-disulfonic acid imidazolium hydrogen sulfate [Im(-SO₃H)₂][HSO₄] IL, an amount of [Im(-SO₃H)₂][Cl](13.47 g, 0.05 mol) was placed in a 20 mL screw top borosilicate glass vial and liquid sulfuric acid (H₂SO₄; 4.95 g, 0.05 mol) was added dropwise. After addition, the reaction mixture was stirred at room temperature for 4 h and then dried under high vacuum for 24 h, giving a brown liquid IL [Im(-SO₃H)₂][HSO₄].

### Example 1-V: Preparation of N,N-Disulfonic Acid Imidazolium Tetrafluoroethane Sulfonate [Im(-SO₃H)₂][TFES] IL

In a 20 mL screw top borosilicate glass vial, equipped with a stir bar, an amount of [Im(-SO₃H)₂][Cl] (13.47 g, 0.05 mol) was placed and then liquid TFESA (9.15 g, 0.05 mol) was added dropwise. After addition, the reaction mixture was stirred at room temperature for 4 h and then dried under high vacuum for 24 h, giving a brown liquid IL [Im(-SO₃H)₂][TFES].

### Example 1-VI: Preparation of N-Methyl, N-Sulfonic Acid Imidazolium Tetrafluoroethane Sulfonate [C₁im-SO₃H][TFES] IL

In a 500 mL round bottom flask, equipped with a stir bar, N-methyl imidazole (8.21 g, 0.10 mol) was reacted with chlorosulfonic acid (12.20 g, 0.104 mol) in dry dichloromethane solvent (40 mL). After addition, the reaction mixture was stirred for 12 h. The dichloromethane solvent was removed under reduced pressure, yielding a white solid. The solid was washed twice with dry diethyl ether and dried under vacuum, yielding a white solid of [C₁im-SO₃] zwitterion type of salt. For the synthesis of [C₁im-SO₃H][TFES] IL, a solid [C₁im-SO₃] zwitterionic salt (8.10 g, 0.05 mol) was placed in a 20 mL screw top borosilicate glass vial and tetrafluoroethanesulfonic acid (TFESA) (9.15 g, 0.05 mol) was added dropwise. After addition of the reactants, the reaction mixture was stirred at room temperature for 4 h, giving a brown liquid IL [C₁im-SO₃H][TFES].

### Example 2. Preparation of Ternary Mixtures of an Ionic Liquid, an Acid, and an Aromatic

### Example 2.1-I Preparation of ([HN₂₂—SO₃H][HSO₄])₁₀-(TFESA)₉₀-(Mesi)₁ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 1 wt % of mesitylene (0.025 g) was added and the reaction mixture was stirred at room temperature for 5 min, giving a liquid double salt IL clathrate ([HN₂₂—SO₃H][HSO₄])₁₀-(TFESA)₉₀-(Mesi)₁.

### Example 2.1-II Preparation of ([HN₂₂—SO₃H][HSO₄])₁₀-(TFESA)₉₀-(Mesitylene)₃ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 3 wt % of mesitylene (0.075 g) was added and the reaction mixture was stirred at room temperature for 5 min, giving a liquid double salt IL clathrate ([HN₂₂—SO₃H] [HSO₄])₁₀-(TFESA)₉₀-(Mesi)₃.

### Example 2.1-III: Preparation of ([HN₂₂—SO₃H][HSO₄])₁₀-(TFESA)₉₀-(Mesi)₃ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([HN₂₂—SO₃H] [HSO₄])₁₀-(TFESA)₉₀-(Mesi)₅.

### Example 2.1-IV: Preparation of ([HN₂₂—SO₃H][HSO₄])₁₀-(TFESA)₉₀-(Mesi)₇ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 7 wt % of mesitylene (0.175 g) was added and the reaction mixture was stirred at room temperature for 5 min, giving a liquid double salt IL clathrate ([HN₂₂—SO₃H][HSO₄])₁₀-(TFESA)₉₀-(Mesi)₇.

### Example 2.1-V: Preparation of ([HN₂₂—SO₃H][HSO₄])₁₀-(TFESA)₉₀-(Mesi)₈ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 8 wt % of mesitylene (0.20 g) was added and the reaction mixture was stirred at room temperature for 5 min, giving a liquid double salt IL clathrate ([HN₂₂—SO₃H][HSO₄])₁₀-(TFESA)₉₀-(Mesi)₃.

### Example 2.1-VI: Preparation of ([HN₂₂—SO₃H][HSO₄])₁₀-(TFESA)₉₀-(Mesi)_{8.5} Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 8.5 wt % of mesitylene (0.213 g) was added and the reaction mixture was stirred at room temperature for 5 min, giving a liquid double salt IL clathrate ([HN₂₂—SO₃H][HSO₄])₁₀-(TFESA)₉₀-(Mesi)₃5.

### Example 2.1-VII: Preparation of ([HN₂₂—SO₃H][HSO₄])₁₀-(TFESA)₉₀-(Mesi)₉ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 9 wt % of mesitylene (0.225 g) was added and the reaction mixture was stirred at room temperature for 5 min, giving a liquid double salt IL clathrate ([HN₂₂—SO₃H] [HSO₄])₁₀-(TFESA)₉₀-(Mesi)₉.

### Example 2.1-VIII: Preparation of ([HN₂₂—SO₃H][HSO₄])₁₀-(TFESA)₉₀-(Mesi)₁₀ Clathrate

In a 20 mL high pressure glass tube, equipped with a stir bar, TFESA (2.25 g) and IL [HN₂₂—SO₃H][HSO₄] (0.25 g) were added at 10:90 wt % and mixed by handshake. After a minute, 10 wt % of mesitylene (0.250 g) was added and the reaction mixture was stirred at room temperature for 5 min, giving a liquid double salt IL clathrate ([HN₂₂—SO₃H][HSO₄])₁₀-(TFESA)₉₀-(Mesi)₁₀.

### Example 2.2-I: Preparation of ([C₁C₄im][HSO₄])₁₀-(TFESA)₉₀-(Mesi)₃ Clathrate

In a 20 mL high pressure glass tube, equipped with a stir bar, TFESA (2.25 g) and IL [C₁C₄im][HSO₄] (0.25 g, commercially available) was added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀-(TFESA)₉₀-(Mesi)₅.

### Example 2.2-II: Preparation of ([C₁C₄im][HSO₄])₁₀-(TFESA)₉₀-(Mesi)_{5.5} Clathrate

In a 20 mL high pressure glass tube, equipped with a stir bar, TFESA (2.25 g) and IL [C₁C₄im][HSO₄] (0.25 g) were added at 10:90 wt % and mixed by handshake. After a minute, 5.5 wt % of mesitylene (0.138 g) was added and the reaction mixture was stirred at room temperature for 5 min, giving a liquid double salt IL clathrate ([C₁C₄im][HSO₄])₁₀-(TFESA)₉₀-(Mesi)_{5.5.}

### Example 2.2-III: Preparation of ([C₁C₄im][HSO₄])₁₀-(TFESA)₉₀-(Mesi)₆ Clathrate

In a 20 mL high pressure glass tube, equipped with a stir bar, TFESA (2.25 g) and IL [C₁C₄im][HSO₄] (0.25 g) were added at 10:90 wt % and mixed by handshake. After a minute, 6 wt % of mesitylene (0.150 g) was added and the reaction mixture was stirred at room temperature for 5 min, giving a liquid double salt IL clathrate ([C₁C₄im][HSO₄])₁₀-(TFESA)₉₀-(Mesi)₆.

### Example 2.2-IV: Preparation of ([C₁C₄im][HSO₄])₁₀-(TFESA)₉₀-(Mesi)₇ Clathrate

In a 20 mL high pressure glass tube, equipped with a stir bar, TFESA (2.25 g) and IL [C₁C₄im][HSO₄] (0.25 g) were added at 10:90 wt % and mixed by handshake. After a minute, 7 wt % of mesitylene (0.175 g) was added and the reaction mixture was stirred at room temperature for 5 min, giving a liquid double salt IL clathrate ([C₁C₄im][HSO₄])₁₀-(TFESA)₉₀-(Mesi)₇.

### Example 2.2-V: Preparation of ([C₁C₄im][HSO₄])₁₀-(TFESA)₉₀-(Mesi)₉ Clathrate

In a 20 mL high pressure glass tube, equipped with a stir bar, TFESA (2.25 g) and IL [C₁C₄im][HSO₄] (0.25 g) were added at 10:90 wt % and mixed by handshake. After a minute, 9 wt % of mesitylene (0.225 g) was added and the reaction mixture was stirred at room temperature for 5 min, giving a liquid double salt IL clathrate ([C₁C₄im][HSO₄])₁₀-(TFESA)₉₀-(Mesi)₉.

### Example 2.3-I: Preparation of ([C₁C₄im][HSO₄])₁₀-(TFMSA)₉₀-(Mesi)₃ Clathrate

In a 20 mL high pressure glass tube, equipped with a stir bar, triflic acid (trifluoroethane sulfonic acid; TFMSA) (2.25 g) and IL [C₁C₄im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀-(TFMSA)₉₀-(Mesi)₅.

### Example 2.3-II: Preparation of ([C₁C₄im][HSO₄])₁₀-(TFMSA)₉₀-(Mesi)₆ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 6 wt % of mesitylene (0.150 g) was added and the reaction mixture was stirred at room temperature for 5 min, giving a liquid double salt IL clathrate ([C₁C₄im][HSO₄])₁₀-(TFMSA)₉₀-(Mesi)₆.

### Example 2.3-III: Preparation of ([C₁C₄im][HSO₄])₁₀-(TFMSA)₉₀-(Mesi)₇ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 7 wt % of mesitylene (0.175 g) was added and the reaction mixture was stirred at room temperature for 5 min, giving a liquid double salt IL clathrate ([C₁C₄im] [HSO₄])₁₀-(TFMSA)₉₀-(Mesi)₇.

### Example 2.4-I: Preparation of ([C₁C₄im][HSO₄])₁₀-(TFMSA)₉₀-(Tol)₄ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 4 wt % of toluene (0.100 g) was added and the reaction mixture was stirred at room temperature for 5 min, giving a liquid double salt IL clathrate ([C₁C₄im][HSO₄])₁₀-(TFMSA)₉₀-(Tol)₄.

### Example 2.4-II: Preparation of ([C₁C₄im][HSO₄])₁₀-(TFMSA)₉₀-(Tol)₆ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 6 wt % of toluene (0.150 g) was added and the reaction mixture was stirred at room temperature for 5 min, giving a liquid double salt IL clathrate ([C₁C₄im][HSO₄])₁₀-(TFMSA)₉₀-(Tol)₆.

### Example 2.5-I: Preparation of ([C₁C₄im][HSO₄])₁₀-(TFMSA)₉₀-(p-Xyl)₄ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 4 wt % of p-xylene (0.100 g) was added and the reaction mixture was stirred at room temperature for 5 min, giving a liquid double salt IL clathrate ([C₁C₄im] [HSO₄])₁₀-(TFMSA)₉₀-(p-Xyl)₄.

### Example 2.5-II: Preparation of ([C₁C₄im][HSO₄])₁₀-(TFMSA)₉₀-(p-Xyl)₆ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 6 wt % of p-xylene (0.150 g) was added and the reaction mixture was stirred at room temperature for 5 min, giving a liquid double salt IL clathrate ([C₁C₄im] [HSO₄])₁₀-(TFMSA)₉₀-(p-Xyl)₆.

### Example 2.6-I: Preparation of ([C₁C₄im][HSO4])₁₀-(TFMSA)₉₀-(Hexamethylbenzene)₂ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 2 wt % of hexamethylbenzene (0.50 g) was added and the reaction mixture was stirred at room temperature for 5 min, giving a liquid double salt IL clathrate ([C₁C₄im][HSO₄])₁₀-(TFMSA)₉₀-(Hexamethylbenzene)₂.

### Example 2.6-II: Preparation of ([C₁C₄im][HSO₄])₁₀-(TFMSA)₉₀-(Hexamethylbenzene)₇ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 7 wt % of hexamethylbenzene (0.175 g) was added and the reaction mixture was stirred at room temperature for 5 min, giving a liquid double salt IL clathrate ([C₁C₄im][HSO₄])₁₀-(TFMSA)₉₀-(Hexamethylbenzene)₇.

### Example 2.6-111: Preparation of ([C₁C₄im][HSO₄])₁₀-(TFMSA)₉₀-(Hexamethylbenzene)₉ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 9 wt % of hexamethylbenzene (0.225 g) was added and the reaction mixture was stirred at room temperature for 5 min, giving a liquid double salt IL clathrate ([C₁C₄im][HSO₄])₁₀-(TFMSA)₉₀-(Hexamethylbenzene)₉.

### Example 2.6-IV: Preparation of ([C₁C₄im][HSO₄])₁₀-(TFMSA)₉₀-(Hexamethylbenzene)₁₀ Clathrate

In a 20 mL high pressure glass tube, equipped with a stir bar, triflic acid (TFMSA) (2.25 g) and IL [C₁C₄im][HSO₄] (0.25 g) were added at 10:90 wt % and mixed by handshake. After a minute, 10 wt % of hexamethylbenzene (0.250 g) was added and the reaction mixture was stirred at room temperature for 5 min, giving a liquid double salt IL clathrate ([C₁C₄im][HSO₄])₁₀-(TFMSA)₉₀-(Hexamethylbenzene)₁₀.

### Example 2.7-I: Preparation of ([C₁C₄im][HSO₄])₁₀-(TFA)₉₀-(Mesi)₃ Clathrate

In a 20 mL high pressure glass tube, equipped with a stir bar, trifluoracetic acid (TFA) (2.25 g) and IL [C₁C₄im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀-(TFA)₉₀-(Mesi)₅.

### Example 2.8-I: Preparation of ([C₁C₄im][HSO₄])₁₀—(H₂SO₄)₉₀-(Mesi)₅ Clathrate

In a 20 mL high pressure glass tube, equipped with a stir bar, H₂SO₄ acid (2.25 g) and IL [C₁C₄im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀—(H₂SO₄)₉₀-(Mesi)₅.

### Example 2.9-I: Preparation of ([C₁C₄im][HSO₄])₁₀-(MSA)₉₀-(Mesi)₅ Clathrate

In a 20 mL high pressure glass tube, equipped with a stir bar, methanesulfonic acid (MSA; 2.25 g) and IL [C₁C₄im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀-(MSA)₉₀-(Mesi)₅.

### Example 2.10-I: Preparation of ([C₁C₄im][HSO₄])₁₀—(HsPO₄)₉₀-(Mesi)₅ Clathrate

In a 20 mL high pressure glass tube, equipped with a stir bar, H₃PO₄ acid (2.25 g) and IL [C₁C₄im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HSO₄])₁₀—(H₃PO₄)₉₀-(Mesi)₅.

### Example 2.11-I: Preparation of ([C₁im-SO₃H][TFES])₁₀-(TFESA)₉₀-(Mesi)₅ Clathrate

In a 20 mL high pressure glass tube, equipped with a stir bar, TFESA (2.25 g) and IL [C₁im-SO₃H][TFES] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁im-SO₃H][TFES])₁₀-(TFESA)₉₀-(Mesi)₅.

### Example 2.11-II: Preparation of ([C₁im-SO₃H][TFES])₁₀-(TFESA)₉₀-(Mesi)₉ Clathrate

In a 20 mL high pressure glass tube, equipped with a stir bar, TFESA (2.25 g) and IL [C₁im-SO₃H][TFES] (0.25 g) were added at 10:90 wt % and mixed by handshake. After a minute, 9 wt % of mesitylene (0.225 g) was added and the reaction mixture was stirred at room temperature for 5 min, giving a liquid double salt IL clathrate ([C₁im-SO₃H][TFES])₁₀-(TFESA)₉₀-(Mesi)₉.

### Example 2.12-I: Preparation of ([C₁C₄im][HFPS])₁₀-(TFMSA)₉₀-(Mesi)₅ Clathrate

In a 20 mL high pressure glass tube, equipped with a stir bar, TFMSA (2.25 g) and IL [C₁C₄im][HFPS] (0.25 g, commercially available) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁C₄im][HFPS])₁₀-(TFMSA)₉₀-(Mesi)₅.

### Example 2.13-I: Preparation of ([C₁C₄im][Tf₂N])₁₀-(TFMSA)₉₀-(p-Xyl)₂ Clathrate

In a 20 mL high pressure glass tube, equipped with a stir bar, TFMSA (2.25 g) and IL [C₁C₄im][Tf₂N] (0.25 g, commercially available) were added at 10:90 wt % and mixed by handshake. After a minute, 2 wt % of p-xylene (0.50 g) was added and the reaction mixture was stirred at room temperature for 5 min, giving a liquid double salt IL clathrate ([C₁C₄im][Tf₂N])₁₀-(TFMSA)₉₀-(p-Xyl)₂.

### Example 2.13-II: Preparation of ([C₁C₄im][Tf₂N])₁₀-(TFMSA)₉₀-(p-Xyl)₄ Clathrate

In a 20 mL high pressure glass tube, equipped with a stir bar, TFMSA (2.25 g) and IL [C₁C₄im][Tf₂N] (0.25 g) were added at 10:90 wt % and mixed by handshake. After a minute, 4 wt % of p-xylene (0.100 g) was added and the reaction mixture was stirred at room temperature for 5 min, giving a liquid double salt IL clathrate ([C₁C₄im][Tf₂N])₁₀-(TFMSA)₉₀-(p-Xyl)₄.

### Example 2.14-I: Preparation of ([C₁C₄im][BF₄])₁₀-(TFMSA)₉₀-(p-Xyl)₂ Clathrate

In a 20 mL high pressure glass tube, equipped with a stir bar, TFMSA (2.25 g) and IL [C₁C₄im][BF₄] (0.25 g, commercially available) were added at 10:90 wt % and mixed by handshake. After a minute, 2 wt % of p-xylene (0.50 g) was added and the reaction mixture was stirred at room temperature for 5 min, giving a liquid double salt IL clathrate ([C₁C₄im][BF₄])₁₀-(TFMSA)₉₀-(p-Xyl)₂.

### Example 2.14-II: Preparation of ([C₁C₄im][BF₄])₁₀-(TFMSA)₉₀-(p-Xyl)₄ Clathrate

In a 20 mL high pressure glass tube, equipped with a stir bar, TFMSA (2.25 g) and IL [C₁C₄im][BF₄] (0.25 g) were added at 10:90 wt % and mixed by handshake. After a minute, 4 wt % of p-xylene (0.100 g) was added and the reaction mixture was stirred at room temperature for 5 min, giving a liquid double salt IL clathrate ([C₁C₄im][BF₄])₁₀-(TFMSA)₉₀-(p-Xyl)₄.

### Example 2.15-I: Preparation of ([Im-(SO₃H)₂][HSO₄])₁₀-(TFESA)₉₀-(Mesi)₉ Clathrate

In a 20 mL high pressure glass tube, equipped with a stir bar, TFESA (2.25 g) and IL [Im-(SO₃H)₂][HSO₄] (0.25 g) were added at 10:90 wt % and mixed by handshake. After a minute, 9 wt % of mesitylene (0.225 g) was added and the reaction mixture was stirred at room temperature for 5 min, giving a liquid double salt IL clathrate ([Im-(SO₃H)₂][HSO₄]₁₀-(TFESA)₉₀-(Mesi)₉.

### Example 2.16-I: Preparation of ([C₁im][HSO₄])₁₀-(TFESA)₉₀-(Mesi)₅ Clathrate

In a 20 mL high pressure glass tube, equipped with a stir bar, TFESA (2.25 g) and IL [C₁im][HSO₄] (0.25 g) were added at 10:90 wt % and mixed by handshake. After a minute, 5 wt % of mesitylene (0.125 g) was added and the reaction mixture was stirred at room temperature for 5 min, giving a liquid double salt IL clathrate ([C₁im][HSO₄]₁₀-(TFESA)₉₀-(Mesi)₅.

### Example 2.16-II: Preparation of ([C₁im][HSO₄])₁₀-(TFESA)₉₀-(Mesi)₆ Clathrate

In a 20 mL high pressure glass tube, equipped with a stir bar, TFESA (2.25 g) and IL [C₁im][HSO₄] (0.25 g) were added at 10:90 wt % and mixed by handshake. After a minute, 6 wt % of mesitylene (0.150 g) was added and the reaction mixture was stirred at room temperature for 5 min, giving a liquid double salt IL clathrate ([C₁im][HSO₄]₁₀-(TFESA)₉₀-(Mesi)₆.

### Example 2.16-III: Preparation of ([C₁im][HSO₄])₁₀-(TFESA)₉₀-(Mesi)₇ Clathrate

In a 20 mL high pressure glass tube, equipped with a stir bar, TFESA (2.25 g) and IL [C₁im][HSO₄] (0.25 g) were added at 10:90 wt % and mixed by handshake. After a minute, 7 wt % of mesitylene (0.175 g) was added and the reaction mixture was stirred at room temperature for 5 min, giving a liquid double salt IL clathrate ([C₁im][HSO₄]₁₀-(TFESA)₉₀-(Mesi)₇.

### Example 2.17-I: Preparation of ([C₁C₄im][HSO₄])₂₀-(TFMSA)₈₀-(Hexamethylbenzene)₂ Clathrate

In a 20 mL high pressure glass tube, equipped with a stir bar, triflic acid (TFMSA) (2.0 g) and IL [C₁C₄im][HSO₄] (0.50 g) were added at 20:80 wt % and mixed by handshake. After a minute, 2 wt % of hexamethylbenzene (0.50 g) was added and the reaction mixture was stirred at room temperature for 5 min, giving a liquid double salt IL clathrate ([C₁C₄im][HSO₄])₂₀-(TFMSA)₈₀-(Hexamethylbenzene)₂.

### Example 2.18-I: Preparation of ([C₁C₄im][HSO₄])₁₀—(H₂SO₄)₉₀-(p-Xyl)₁ Clathrate

In a 20 mL high pressure glass tube, equipped with a stir bar, H₂SO₄ (2.25 g) and [C₁C₄im][HSO₄] IL (0.25 g) were mixed at 10:90 wt % and stirred at 5° C. for 10 min. Later, 1 wt % of p-xylene (0.025 g) was added and the reaction mixture was stirred for 5 min, giving a liquid double salt IL clathrate ([C₁C₄im][HSO₄])₁₀—(H₂SO₄)₉₀-(p-Xyl)₁.

Example 3. Preparation of ternary mixtures of a base, an aromatic, and an acid. These Examples demonstrate the in situ formation of an ionic liquid in the ternary mixture. That is, the base component and the acid component in the ternary mixture react to form the ionic liquid in situ.

### Example 3.1-I: Preparation of (C₁im)₁₀-(TFMSA)₉₀-(p-Xyl)₁ Clathrate

In a 20 mL high pressure glass tube, equipped with a stir bar, triflic acid (TFMSA) (2.25 g) and 1-methylimidazole (C₁im; 0.25 g) were mixed at 10:90 wt % and stirred at 8° C. for 10 min. Later, 1 wt % of p-xylene (0.025 g) was added and the reaction mixture was stirred for 5 min, giving a liquid double salt IL clathrate (C₁im)₁₀-(TFMSA)₉₀-(p-Xyl)₁.

### Example 3.2-I: Preparation of (Imidazole)₁₀-(TFMSA)₉₀-(p-Xyl)_{0.5} Clathrate

In a 20 mL high pressure glass tube, equipped with a stir bar, triflic acid (TFMSA) (2.25 g) and imidazole (0.25 g) were mixed at 10:90 wt % and stirred at 8° C. for 10 min. Later, 0.5 wt % of p-xylene (0.013 g) was added and the reaction mixture was stirred for 5 min, giving a liquid double salt IL clathrate (Imidazole)₁₀-(TFMSA)₉₀-(p-Xyl)_{0.5}.

### Example 3.2-II: Preparation of (Imidazole)₁₀-(TFMSA)₉₀-(p-Xyl)₁ Clathrate

In a 20 mL high pressure glass tube, equipped with a stir bar, triflic acid (TFMSA) (2.25 g) and imidazole (0.25 g) were mixed at 10:90 wt % and stirred at 8° C. for 10 min. Later, 1 wt % of p-xylene (0.025 g) was added and the reaction mixture was stirred for 5 min, giving a liquid double salt IL clathrate (Imidazole)₁₀-(TFMSA)₉₀-(p-Xyl)₁.

### Example 3.3-I: Preparation of (C₁im)₁₀-(H₂SO₄)₉₀-(p-Xyl)₁ Clathrate

In a 20 mL high pressure glass tube, equipped with a stir bar, H₂SO₄ (2.25 g) and 1-methylimidazole (C₁im; 0.25 g) were mixed at 10:90 wt % and stirred at 8° C. for 10 min. Later, 1 wt % of p-xylene (0.025 g) was added and the reaction mixture was stirred for 5 min, giving a liquid double salt IL clathrate (C₁im)₁₀-(H₂SO₄)₉₀-(p-Xyl)₁.

### Example 4. Isobutane/Butene Alkylation Apparatus and Procedure

Isobutane/2-butene alkylations were performed in a 20 mL high pressure glass reactor as shown in FIG. 3. Cooling was provided by a recirculating chiller using ethylene glycol. The isobutane and 2-butene were premixed at 90:10 wt ratio and collected in ISCO pump in liquid phase. The gas phase (and also unreacted liquid butene converted into gas phase) was collected in a gas sampling bag (1 Lit). The products were analyzed offline by gas chromatography (GC), equipped with a flame ionization detector, and a DB-5 100 m column (J&W Scientific). Helium was used as the GC carrier gas and as the flame ionization detector (FID) makeup gas. The analysis conditions were: split ratio=50:1, injector temperature=280° C., detector temperature=300° C. carrier gas flow rate=20 sccm. The temperature program for GC analysis was as follows: initial column temperature 30° C./hold for 15 min, 0.5° C./min to 100° C., then 5° C./min to 300° C./hold for 15 min. An alkylate reference standard (Supelco) allowed identification of the trimethylpentanes (TMP) and dimethylhexanes (DMH). The GC area percent was equated to weight percent since all hydrocarbons in the reactor effluent had response factors close to unity. The combined mass of TMP and DMH is referred to as the “alkylate product”. The gas phase was also analyzed by GC and the butene conversion was calculated.

All experiments were performed in batch. A typical experiment began with the addition of the catalyst into the 20 mL high pressure glass reactor. The reactor was sealed and cooled to the desired temperature. The desired amount of hydrocarbon feed was pumped into the reactor at a flow rate of 1 mL/min while stirring the liquid phase. After a certain reaction time (1-5 min), stirring was stopped and the gas phase was collected in the gas sampling bag quickly. The unreacted liquid hydrocarbon was converted into the gas phase by waiting for approximately 1-3 min and collecting in the same sampling bag.

The results are shown in Table 1.

TABLE 1
Isobutane Alkylation Results.

| | | | | Temp/ | |
|---|---|---|---|---|---|
| | | Cat. | Amt | time | |
| S. | | Amt | of C₄ | (° C./ | Conv |
| N. | Catalyst | (g) | Feed | min) | (%) |
| 1 | DuPont H₂SO₄ (Fresh + Spent; 94%) | 2.5 | 2 mL | 8° C./5 | >99.9 |
| 2 | TFESA | 1.0 | 2 mL | 8° C./5 | 97.4 |
| 4 | ([HN₂₂—SO₃H][HSO₄])₁₀-(TFESA)₉₀-(Mesi)₁ | | | | >99.9 |
| 5 | ([HN₂₂—SO₃H][HSO₄])₁₀-(TFESA)₉₀₋(Mesi)₃ | | | | |
| 6 | ([HN₂₂—SO₃H][HSO₄])₁₀-(TFESA)₉₀-(Mesi)₅ | | | | |
| 7 | ([HN₂₂—SO₃H][HSO₄])₁₀-(TFESA)₉₀-(Mesi)₇ | | | | |
| 8 | ([HN₂₂—SO₃H][HSO₄])₁₀-(TFESA)₉₀-(Mesi)_{8.5} | | | | |
| 9 | ([HN₂₂—SO₃H][HSO₄])₁₀-(TFESA)₉₀-(Mesi)₉ | | | | |
| 10 | ([HN₂₂—SO₃H][HSO₄])₁₀-(TFESA)₉₀-(Mesi)₁₀ | | | | |
| 11 | ([HN₂₂—SO₃H][HSO₄])₁₀-(TFESA)₉₀-(Mesi)₉ | 0.5 | 2 mL | 8° C./5 | >99.9 |
| 12 | | 1.0 | | | |
| 13 | | 1.5 | | | |
| 14 | | 2.0 | | | |
| 15 | | 2.5 | | | |
| 16 | | 2.5 | 2 mL | 0° C./5 | >99.9 |
| 17 | | | | 3° C./5 | |
| 18 | | | | 5° C./5 | |
| 19 | | | | 8° C./5 | |
| 20 | | | | 8° C./1 | |
| 21 | | | | 15° C./5 | |
| 22 | | | | 20° C./5 | |
| 23 | | 1.0 | 2 mL | 20° C./1 | |
| 24 | ([HN₂₂—SO₃H][HSO₄])₁₀-(TFESA)₉₀-(Mesi)₉ | 2.5 | 5 mL | 8° C./1 | >99.9 |
| 25 | ([HN₂₂—SO₃H][HSO₄])₁₀-(TFESA)₉₀-(Mesi)_{8.7} | 2.5 | 5 mL | 8° C./1 | >99.9 |
| 27 | ([C₁C₄im][HSO₄])₁₀-(TFESA)₉₀₋(Mesi)₉ | | 2 mL | 8° C./1 | 92.0 |
| 28 | ([C₁C₄im][HSO₄])₁₀-(TFESA)₉₀₋(Mesi)₇ | | 2 mL | 8° C./5 | >99.9 |
| 29 | ([C₁C₄im][HSO₄])₁₀(TFESA)₉₀-(Mesi)₆ | | 2 mL | | >99.9 |
| 30 | ([C₁C₄im][HSO₄])₁₀-(TFESA)₉₀₋(Mesi)_{5.5} | | 2 mL | | >99.9 |
| 31 | ([C₁C₄im][HSO₄])₁₀-(TFESA)₉₀₋(Mesi)₅ | | 2 mL | | >99.9 |
| 32 | ([C₁C₄im][HSO₄])₁₀-(TFESA)₉₀₋(Mesi)₅ | 2.5 | 2 mL | 5° C./5 | >99.9 |
| 33 | ([C₁C₄im][HSO₄])₁₀-(TFESA)₉₀₋(Mesi)₅ | 2.5 | 5 mL | 8° C./5 | >99.9 |
| 34 | TFMSA | 2.5 | 5 mL | 5° C./5 | >99.9 |
| 35 | ([C₁C₄im][HSO₄])₁₀-(TFMSA)₉₀₋(Mesi)₅ | 2.5 | 2 mL | 8° C./5 | >99.9 |
| 36 | ([C₁C₄im][HSO₄])₁₀-(TFMSA)₉₀₋(Mesi)₆ | 2.5 | 2 mL | 8° C./5 | |
| 37 | ([C₁C₄im][HSO₄])₁₀-(TFMSA)₉₀₋(Mesi)₇ | 2.5 | 2 mL | 8° C./5 | |
| 38 | ([C₁C₄im][HSO₄])₁₀-(TFMSA)₉₀₋(Mesi)₆ | 2.5 | 5 mL | 8° C./5 | >99.9 |
| 39 | ([C₁C₄im][HSO₄])₁₀—(H₂SO4)₉₀-(Mesi)₅ | 2.5 | 2 mL | 8° C./5 | >99.9 |
| 44 | ([C₁C₄im][HSO₄])₁₀-(TFMSA)₉₀₋(Mesi)₆ | 2.5 | 5 mL | 8° C./5 | >99.9 |
| 45 | ([C₁C₄im][HSO₄])₁₀-(TFMSA)₉₀₋(Mesi)₆ | 2.5 | 5 mL | 5° C./5 | >99.9 |
| 46 | ([C₁C₄im][HSO₄])₁₀-(TFMSA)₉₀₋(Tol)₆ | 2.5 | 5 mL | 5° C./5 | >99.9 |
| 47 | ([C₁C₄im][HSO₄])₁₀-(TFMSA)₉₀₋(Tol)₄ | 2.5 | 5 mL | 5° C./5 | >99.9 |
| 48 | ([C₁C₄im][HSO₄])₁₀-(TFMSA)₉₀₋(p-Xyl)₆ | 2.5 | 5 mL | 5° C./5 | >99.9 |
| 49 | ([C₁C₄im][HSO₄])₁₀-(TFMSA)₉₀₋(p-Xyl)₅ | 2.5 | 5 mL | 5° C./5 | >99.9 |
| 50 | ([C₁C₄im][HSO₄])₁₀-(TFMSA)₉₀₋(p-Xyl)₄ | 2.5 | 5 mL | 5° C./5 | >99.9 |
| 51 | ([C₁C₄im][HSO₄])₁₀-(TFMSA)₉₀- | 2.5 | 5 mL | 5° C./5 | >99.9 |
| | (Hexamethylbenzene)₂ | | | | |
| 52 | ([C₁C₄im][HSO₄])₁₀-(TFMSA)₉₀- | 2.5 | 5 mL | 5° C./5 | >99.9 |
| | (Hexamethylbenzene)₇ | | | | |
| 53 | ([C₁C₄im][HSO₄])₁₀-(TFMSA)₉₀- | 2.5 | 5 mL | 5° C./5 | >99.9 |
| | (Hexamethylbenzene)₉ | | | | |
| 54 | ([C₁C₄im][HSO₄])₁₀-(TFMSA)₉₀- | 2.5 | 5 mL | 5° C./5 | >99.9 |
| | (Hexamethylbenzene)₁₀ | | | | |
| 55 | ([C₁C₄im][HSO₄])₁₀-(TFMSA)₉₀- | | | | |
| | (Fluorobenzene)₄ | | | | |
| 57 | ([C₁im-SO₃H][TFES])₁₀-(TFESA)₉₀-(Mesi)₉ | 2.5 | 2 mL | 8° C./1 | >99.9 |
| 58 | ([C₁im-SO₃H][TFES])₁₀-(TFESA)₉₀-(Mesi)₅ | 2.5 | 2 mL | 8° C./1 | |
| 59 | ([C₁C₄im][HFPS])₁₀-(TFESA)₉₀₋(Mesi)₅ | 2.5 | 2 mL | 8° C./5 | >99.9 |
| 60 | ([C₁C₄im][Tf₂N])₁₀-(TFMSA)₉₀₋(p-Xyl)₄ | 2.5 | 5 mL | 8° C./5 | >99.9 |
| 61 | ([C₁C₄im][Tf₂N])₁₀-(TFMSA)₉₀₋(p-Xyl)₂ | 2.5 | 5 mL | 8° C./5 | >99.9 |
| 63 | ([C₁C₄im][BF₄])₁₀-(TFMSA)₉₀₋(p-Xyl)₄ | 2.5 | 5 mL | 8° C./5 | 72.5 |
| 64 | ([C₁C₄im][BF₄])₁₀-(TFMSA)₉₀-(p-Xyl)₂ | 2.5 | 5 mL | 8° C./5 | 80.0 |
| 66 | ([Im-(SO₃H)₂][HSO₄])₁₀-(TFESA)₉₀₋(Mesi)₉ | 2.5 | 2 mL | 8° C./1 | >99.9 |
| 69 | ([C₁im][HSO₄])₁₀-(TFESA)₉₀₋(Mesi)₆ | 2.5 | 2 mL | 8° C./5 | >99.9 |
| 70 | ([C₁im][HSO₄])₁₀-(TFESA)₉₀₋(Mesi)₇ | 2.5 | 2 mL | 8° C./5 | >99.9 |
| 77 | ([C₁C₄im][HSO₄])₂₀-(TFMSA)₈₀- | 2.5 | 5 mL | 5° C./5 | >99.9 |
| | (Hexamethylbenzene)₂ | | | | |
| 78 | TFMSA | 2.5 | 5 mL | 5° C./5 | >99.9 |
| 80 | (N-methyl imidazole)₁₀-(TFMSA)₉₀-(p-Xyl)₁ | 2.5 | 5 mL | 5° C./5 | >99.9 |
| 82 | (Imidazole)₁₀-(TFMSA)₉₀-(p-Xyl)₂ | 2.5 | 5 mL | 5° C./5 | >99.9 |
| 83 | (Imidazole)₁₀-(TFMSA)₉₀₋(p-Xyl)₁ | 2.5 | 5 mL | 5° C./5 | >99.9 |
| 84 | H₂SO₄ (Fresh) | 2.5 | 5 mL | 5° C./5 | >99.9 |
| | Conventional [HN₂₂₂][Al₂Cl₇] | 2.3 | 2 mL | 8° C./20 | >99.9 |
| | [HN₂₂₂][Al₂Cl₇]-10 mol % CuCl | 1.2 | 2 mL | 8° C./5 | >99.9 |
| | | 2.3 | 2 mL | 8° C./5 | >99.9 |

| | Liquid Product Selectivity (%) | | | | | | | | |
|---|---|---|---|---|---|---|---|---|---|
| S. | | | | | | | | C₁₂⁺ | T/D Ratio by |
| N. | C₅ | C₆ | C₇ | C₈ | C₉ | C₁₀ | C₁₁ | Other | DuPont Method |
| 1 | 1.5 | 3.4 | 4.5 | 52.9 | 12.6 | 8.3 | 7.0 | 6.8 | 7.37 |
| 2 | 11.1 | 8.0 | 7.6 | 41.2 | 17 | 8.0 | 3.8 | 1.7 | 3.80 |
| 4 | 7.6 | 5.9 | 6.6 | 53.9 | 14.2 | 4.4 | 3.8 | 1.3 | 4.45 |
| 5 | 5.1 | 4.4 | 5.6 | 64.9 | 9.0 | 3.7 | 4.3 | 1.2 | 5.92 |
| 6 | 3.1 | 3.2 | 4.6 | 76.0 | 5.0 | 1.9 | 3.2 | 1.0 | 8.52 |
| 7 | 1.5 | 2.0 | 3.1 | 84.0 | 1.9 | 1.2 | 3.1 | 0.8 | 13.92 |
| 8 | 1.3 | 1.9 | 3.0 | 84.9 | 1.6 | 1.2 | 2.9 | 1.3 | 15.07 |
| 9 | 1.3 | 1.9 | 2.8 | 86.5 | 1.7 | 1.0 | 3.4 | 1.4 | 15.80 |
| 10 | 0.3 | 0.3 | 2.0 | 67.4 | 0.2 | 0.5 | 1.1 | 26.6* | 29.04 |
| 11 | 2.2 | 3.5 | 4.5 | 65.5 | 6.9 | 5.6 | 7.0 | 3.0 | 10.94 |
| 12 | 2.1 | 2.8 | 3.7 | 78.4 | 3.4 | 1.8 | 4.2 | 3.0 | 13.07 |
| 13 | 1.7 | 2.4 | 3.7 | 82.6 | 2.5 | 1.5 | 4.1 | 1.5 | 13.98 |
| 14 | 1.2 | 1.8 | 3.1 | 85.4 | 1.0 | 0.5 | 2.7 | 3.5 | 16.03 |
| 15 | 1.3 | 1.9 | 2.8 | 86.5 | 1.7 | 1.0 | 3.4 | 1.4 | 15.80 |
| 16 | 1.8 | 3.1 | 4.3 | 58.6 | 8.8 | 6.8 | 8.9 | 7.6 | 10.50 |
| 17 | 1.3 | 2.0 | 3.2 | 81.4 | 2.0 | 1.4 | 4.2 | 4.5 | 16.83 |
| 18 | 1.0 | 1.6 | 3.0 | 87.2 | 1.0 | 0.6 | 3.1 | 1.6 | 18.13 |
| 19 | 1.3 | 1.9 | 2.8 | 86.5 | 1.7 | 1.0 | 3.4 | 1.4 | 15.80 |
| 20 | 1.1 | 1.9 | 3.4 | 86.7 | 1.6 | 0.7 | 3.1 | 1.5 | 16.37 |
| 21 | 1.7 | 2.2 | 3.4 | 84.6 | 2.5 | 1.3 | 2.9 | 1.3 | 12.76 |
| 22 | 2.3 | 2.9 | 4.3 | 78.4 | 5.4 | 1.9 | 3.6 | 0.8 | 9.72 |
| 23 | 1.3 | 2.5 | 3.7 | 76.2 | 4.8 | 2.9 | 5.3 | 3.0 | 10.78 |
| 24 | 0.9 | 1.3 | 2.6 | 88.6 | 0.6 | 0.5 | 2.3 | 3.1 | 20.42 |
| 25 | 1.2 | 1.8 | 3.1 | 87.3 | 1.0 | 0.6 | 2.9 | 2.2 | 17.85 |
| 27 | 0 | 0 | 0.3 | 30.1 | 0 | 0.8 | 0.2 | 0.2 + | 41.60 |
| | | | | | | | | 68.4* | |
| 28 | 0.4 | 0.5 | 1.3 | 64.3 | 0.5 | 0.4 | 1.6 | 0.7 + | 25.35 |
| | | | | | | | | 30.3* | |
| 29 | 0.8 | 1.3 | 2.0 | 82.9 | 0.7 | 0.5 | 2.2 | 1.0 + | 21.10 |
| | | | | | | | | 8.5* | |
| 30 | 0.9 | 1.6 | 2.5 | 87.5 | 1.4 | 0.9 | 3.2 | 1.9 | 18.52 |
| 31 | 1.1 | 1.9 | 2.4 | 87.9 | 1.5 | 1.1 | 2.7 | 1.3 | 17.90 |
| 32 | 1.3 | 2.0 | 2.7 | 84.7 | 1.9 | 1.6 | 3.9 | 2.0 | 17.13 |
| 33 | 1.1 | 1.8 | 2.5 | 88.5 | 1.4 | 0.7 | 2.8 | 1.3 | 17.85 |
| 34 | 13.0 | 7.3 | 8.3 | 47.6 | 17.1 | 3.6 | 2.4 | 0.6 | 2.6 |
| 35 | 1.9 | 1.9 | 2.8 | 86.6 | 2.5 | 1.0 | 2.8 | 0.6 | 12.68 |
| 36 | 1.2 | 1.5 | 2.2 | 89.0 | 1.3 | 0.5 | 3.0 | 1.3 | 16.15 |
| 37 | 0.4 | 0.5 | 1.2 | 86.3 | 0.0 | 0.0 | 1.5 | 10.3 | 22.32 |
| 38 | 1.0 | 1.1 | 1.8 | 91.3 | 0.7 | 0.3 | 2.4 | 1.4 | 19.78 |
| 39 | 1.9 | 4.8 | 5.2 | 54.2 | 11.7 | 7.9 | 6.6 | 3.7 | 6.92 |
| 44 | 1.0 | 1.1 | 1.8 | 91.3 | 0.7 | 0.3 | 2.4 | 1.4 | 19.78 |
| 45 | 0.7 | 0.6 | 1.6 | 93.0 | 0.4 | 0.3 | 2.3 | 1.2 | 22.40 |
| 46 | 0.2 | 0.2 | 0.2 | 72.9 | 0.0 | 0.0 | 0.3 | 0.3 + | 54.07 |
| | | | | | | | | 26.0* | |
| 47 | 0.5 | 0.5 | 1.3 | 90.9 | 0.0 | 0.2 | 1.2 | 0.5 + | 24.96 |
| | | | | | | | | 4.8* | |
| 48 | 0.4 | 0.4 | 1.1 | 86.3 | 0.0 | 0.0 | 0.8 | 0.5 + | 27.41 |
| | | | | | | | | 10.5* | |
| 49 | 0.6 | 0.9 | 1.4 | 90.5 | 0.2 | 0.2 | 1.8 | 0.6 + | 23.71 |
| | | | | | | | | 3.2* | |
| 50 | 0.9 | 1.0 | 1.8 | 92.6 | 0.6 | 0.4 | 2.1 | 0.6 | 21.14 |
| 51 | 1.3 | 1.5 | 2.3 | 89.3 | 1.5 | 0.7 | 2.6 | 0.8 | 15.63 |
| 52 | 0.6 | 0.6 | 1.1 | 94.4 | 0.3 | 0.2 | 1.5 | 1.2 | 29.40 |
| 53 | 0.5 | 0.3 | 0.8 | 92.2 | 0.3 | 0.2 | 0.9 | 0.7 + | 40.0 |
| | | | | | | | | 4.0* | |
| 54 | 0.6 | 0.2 | 0.4 | 89.5 | 0.3 | 0.0 | 0.7 | 0.5 + | 61.57 |
| | | | | | | | | 7.9* | |
| 55 | | | | | | | | | |
| 57 | 0.3 | 0.3 | 1.6 | 50.0 | 0.4 | 0.2 | 5.5 | 0.2 + | 28.10 |
| | | | | | | | | 41.5* | |
| 58 | 2.3 | 2.8 | 4.4 | 81.7 | 3.2 | 1.5 | 3.3 | 0.8 | 11.18 |
| 59 | 1.6 | 2.1 | 2.8 | 73.2 | 2.5 | 1.0 | 7.4 | 9.3* | 12.18 |
| 60 | 0.2 | 0.2 | 0.4 | 68.9 | 0.0 | 0.0 | 0.3 | 0.5 + | 36.11 |
| | | | | | | | | 29.9* | |
| 61 | 1.8 | 1.7 | 2.5 | 87.9 | 0.7 | 0.3 | 2.2 | 3.0* | 8.84 |
| 63 | 0.5 | 0.6 | 0.8 | 67.3 | 1.0 | 0.8 | 1.7 | 0.7 + | 16.79 |
| | | | | | | | | 26.7* | |
| 64 | 1.2 | 1.4 | 1.9 | 84.7 | 1.1 | 0.6 | 2.0 | 0.3 + | 11.81 |
| | | | | | | | | 6.9* | |
| 66 | 1.3 | 1.5 | 7.0 | 85.6 | 1.1 | 0.5 | 2.0 | 0.9 | 16.86 |
| 69 | 1.5 | 2.5 | 3.3 | 78.2 | 2.5 | 2.5 | 5.6 | 3.6 | 14.40 |
| 70 | 0.5 | 0.8 | 1.8 | 81.8 | 0.5 | 0.5 | 2.5 | 11.6 | 20.33 |
| 77 | 0.9 | 0.9 | 1.9 | 90.9 | 0.0 | 0.6 | 2.0 | 2.6 | 21.55 |
| 78 | 13.0 | 7.3 | 8.3 | 47.6 | 17.1 | 3.6 | 2.4 | 0.6 | 2.6 |
| 80 | 1.7 | 2.4 | 3.3 | 67.6 | 2.1 | 2.6 | 5.5 | 3.8 + | 11.92 |
| | | | | | | | | 10.8* | |
| 82 | 1.6 | 2.4 | 3.1 | 53.3 | 2.7 | 3.2 | 5.9 | 4.1 + | 10.79 |
| | | | | | | | | 23.8* | |
| 83 | 1.1 | 2.3 | 3.4 | 62.6 | 3.0 | 3.7 | 6.9 | 4.7 + | 10.85 |
| | | | | | | | | 12.2* | |
| 84 | 2.8 | 4.7 | 5.2 | 55.3 | 14.9 | 7.9 | 5.1 | 4.2 | 6.64 |
| | 18.0 | 12.6 | 14.0 | 29.2 | 16.2 | 6.3 | 1.8 | 0.5 | 0.4 |
| | 20.8 | 12.7 | 11.2 | 36.2 | 12.9 | 4.1 | 1.6 | 0.4 | 1.59 |
| | 15.2 | 10.6 | 9.3 | 42.2 | 3.9 | 7.9 | 0 | 0 | 3.21 |

Reaction condition: Catalyst, isobutene-2-butene premixed liquid {2 or 5 mL, 10 wt. % of 2-butene}, T = ° C., time = min, P = 2 bar.
Note:
TFESA = Tetrafluoroethanesulfonic acid (HCF₂CF₂SO₃H);
TFMSA = Triflic acid (CF₂SO₃H);
Mesi = Mesitylene;
p-Xyl = p-Xylene;
o-Xyl = o-Xylene;
TFA = Triflouroacetic acid;
*Also contains mesitylene alkylated product

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. An alkylation catalyst composition comprising an acid, an aromatic, and a third component, wherein the third component is selected from a group consisting of a base capable of forming an ionic liquid with the acid, and an ionic liquid.

2. The composition of claim 1, wherein the aromatic is an unsubstituted or substituted monocyclic aromatic.

3. The composition of claim 2, wherein the aromatic has formula C₆R₆, wherein each R is independently selected from the group consisting of hydrogen, a halogen, and an unsubstituted or substituted alkyl group.

4. The composition of claim 3, wherein the aromatic is selected from the group consisting of benzene, toluene, xylenes, mesitylene, hexamethylbenzene, and a halogenated benzene.

5. The composition of claim 1, wherein the aromatic is present at an amount in a range of from 1 wt % to 25 wt %.

6. The composition of claim 1, wherein the acid is a mineral acid, a sulfonic acid, or a carboxylic acid.

7. The composition of claim 6, wherein the acid is the sulfonic acid.

8. The composition of claim 7, wherein the sulfonic acid has formula R—SO₃H, wherein R is an unsubstituted alkyl group or a substituted alkyl group.

9. The composition of claim 7, wherein the sulfonic acid is triflic acid, tetrafluoroethanesulfonic acid, or hexafluoropropanesulfonic acid.

10. The composition of claim 7, wherein the acid is not H₂SO₄.

11. The composition of claim 1, wherein the third component is the ionic liquid and the ionic liquid comprises a cation selected from an ammonium, an imidazolium, a sulfonium, a phosphonium, and a pyridinium.

12. The composition of claim 11, wherein the ionic liquid comprises an anion selected from a sulfonate, a carboxylate, [HSO₄]⁻, dicyanamide, and an inorganic anion.

13. The composition of claim 11, wherein the ionic liquid comprises a cation selected from an ammonium and an imidazolium and the anion is [HSO₄]⁻.

14. The composition of claim 11, wherein the ionic liquid is a binary ionic liquid comprising a single type of cation and a single type of anion.

15. The composition of claim 1, wherein the third component is the base and the base is selected from an ammonia, an imidazole, and a pyridine.

16. The composition of claim 1, wherein the third component is present at an amount in a range of from 1 wt % to 30 wt % and the acid is present at an amount in a range of from 99 wt % to 70 wt %.

17. An alkylation process comprising combining the alkylation catalyst composition of claim 1 with a feedstock under conditions to produce an alkylate product for a motor fuel additive.

18. The alkylation process of claim 17, wherein the feedstock comprises an isoalkane and an olefin.

19. The alkylation process of claim 18, wherein the feedstock comprises isobutane and butene.

20. The alkylation process of claim 17, characterized by a conversion of at least 95%, a C8 selectivity of at least 75%, a T/D ratio of at least 10, or combinations thereof.

21. The alkylate product produced by the process of claim 17.

22. An alkylate product comprising branched alkanes comprising trimethylpentane and dimethylhexane and having a percentage of C8 alkanes of at least 95% and a T/D ratio of at least 10.

23. The alkylate product of claim 22 combined with gasoline.
